(19) United States
(12) Patent Application Publication
(10) Pub. No.: US 2005/0199249 A1
(43) Pub. Date: Sep. 15, 2005

(54) Apparatus and method for incision-free vaginal prolapse repair

(76) Inventor: Mickey M. Karram, Cincinnati, OH (US)

Correspondence Address: CHITTARANJAN N. NIRMEL;c/o Nirmel & Associates, Suite 600, 1717 K ST., N.W., WASHINGTON, DC, 20036, US

(21) Appl. No.: 11/064,342

(22) Filed: Feb. 23, 2005

Related U.S. Application Data
(60) Provisional application No. 60/553,315, filed on Mar. 15, 2004.

(51) Int. Cl. A61B 17/04

(52) U.S. Cl. 128/885

(57) ABSTRACT

In a preferred application, e.g., the repair of vaginal prolapse after relocation of the vagina and any organs displaced by the prolapse, corrective surgery is initiated by applying a hollow tubular element, formed to forcibly insert a barbed anchor attached to a distal end of a first length of suture, without any incision, from the inside of the vagina through the vaginal wall (the supported tissue) into selected support tissue within a patient's pelvis. This involves puncturing and thus locally severe physical distressing of both the supported tissue and the support tissue. The barbed anchor is left in the support tissue as the tubular element is then withdrawn from the support tissue and out of the vagina, leaving the proximate end portion of the suture extending through the vaginal wall into the vagina. A second such anchor, with a second length of suture attached thereto, is similarly inserted adjacent to the first anchor. The proximate end portions of the sutures are tied to each other inside the vagina, to thereby secure the vaginal wall to the support tissue with corresponding punctures formed in each by the insertions of the two anchors being thereby held in respective, precisely aligned, intimate contact during healing. This results in a pair of fused scars that cooperate to permanently bond the vaginal wall locally to the support tissue. If the sutures and/or the anchors are made of absorbable material they will all eventually disappear and the fused scars will provide the permanent bonding. If the anchors are made of non-absorbable material they may remain where located. A plurality of such paired fused-scar bonds may be generated, at the surgeon's discretion, to ensure adequate support for the repaired vagina. The apparatus and method can be readily adapted to similarly effect deliberate, local, beneficial bonding between other adjacent living tissues in a patient.

This application claims priority to copending U.S. Provisional Patent Application Ser. No. 60/553,315 of Mickey M. KARRAM, titled “APPARATUS AND METHOD OF REATTACHING PROLAPSED ORGAN WITH BARB-ANCHORED ABSORBABLE SUTURES”, filed Mar. 15, 2004.

### FIELD OF THE INVENTION

This invention relates to an apparatus and a method for permanently repairing vaginal prolapse without making incisions. More particularly, the invention relates to an apparatus and a method by which absorbable or nonabsorbable barbed anchors with attached sutures are implanted through an unopened female patient's relocated vagina into selected support tissue without forming any vaginal or abdominal incisions, to generate deliberately fused scarification locally between the vaginal wall tissue and the support tissue for permanent bonding therebetween.

### BACKGROUND OF THE RELATED ART

Females commonly suffer from pelvic organ prolapse, which results from the breakdown of support structures within the pelvis. This results in the protrusion of vaginal tissue outside the vaginal opening. This can be secondary to protrusion of the anterior vaginal wall (cystocele), protrusion of the posterior vaginal wall (rectocele or enterocele), or protrusion of the uterus or top of the vaginal wall (uterine or vault prolapse). In more severe cases these can occur in combination, leading to complete eversion of the vagina. Such a problem may arise from a variety of causes such as accidental trauma, surgery, weakening due to age or disease, or the like.

The prolapse can result in eversion of the vaginal vault, and may result in physical dependency of displaced organs within the everted vaginal vault either partially or totally dependent outside of the pelvis. This can subject the affected female to significant pain and discomfort and severe functional derangements of the bladder and bowel, as well as physical incapacity and the risk of serious physical harm. The remedy is to return the displaced organs and vagina to their normal dispositions relative to each other within the patient's pelvis, and to then ensure that the relocation is safely and durably maintained by permanent attachment of the vaginal tissue to durable support structures. It is desirable that the remedial procedure should subject the patient only to minimal pain and risk of infection, bleeding, or injury to surrounding structures. The healing should be quick, the overall cost should be low, and the cure should be long-lasting.

Currently practiced procedures to remedy vaginal prolapse problems involve invasive surgery requiring general anasthesia and prolonged operative time. The patient also has to cope with considerable physical pain during the healing process, and risks exposure to various complications and risks incidental to invasive surgery. Furthermore, even successful surgery of this kind leaves the patient with at least some external or vaginal scars, a factor that is aggravated if additional surgery is subsequently required for any reason. These known procedures also require significant surgical expertise in the area of pelvic reconstructive surgery. The surgery and follow-on care during the patient's recovery both tend to be relatively expensive and are particularly burdensome for women due to pain, vaginal discharge or bleeding, and the prolonged time required for bladder and bowel function to return. Simpler, less invasive, less expensive and safer solutions, therefore, need to be developed.

In the related field of surgery to correct female incontinence, for example, various solutions have been proposed that employ non-incisional techniques to implant anchors for slings and sutures to support a patient's urethra.

U.S. Pat. No. 6,595,911, to LoVuolo, titled “Method and Device for Anchor Implantation and Support of Bodily Structures”, teaches a device comprising a sharp-pointed delivery needle that can be curved, a stylet that is slidable within the needle, and a suture-support formed by an implantable anchor toggle to which are attached elongate first and second sutures. The anchor toggle is initially located within the distal end of the needle, from which it is forced out and into selected support tissue by a pushing force applied via the stylet. The method of using the device requires the formation of two incisions through the vaginal wall to allow the surgeon to insert the needle through each to sequentially locate respective anchor toggles into selected support tissue in the abdominal wall. The sutures from the two toggles are then tied to each other behind and outside of the vagina as desired to support the patient's urethra.

U.S. Pat. No. 6,334,446, to Beyar, titled “Medical Sling Procedures and Anchor Insertion Methods and Devices”, teaches a device with a strongly graspable handle and a curved structure to pneumatically force a staple element into bone to thereby anchor a sling to provide support, for example, for a patient's urethra. One type of staple taught herein has a sharp pointed front end and rearwardly inclined barbs, with a transverse hole through which a length of suture extends out on both sides of the staple body. The paired suture lengths from each of two staples are then disposed ipsilaterally on opposite sides of the urethra and tied to cooperatively support the same. A variety of staples are disclosed, for forcible insertion into bone. Some are clearly made of relatively hard metal, e.g., stainless steel, and others comprise comparably strong but flexible alloy.

A device sold under the name “RAPIDLOC™ Repair System” by the Mitek Products Division of Ethicon, a Johnson & Johnson Company, USA, employs a curved needle formed to have a distal side cutout, that is used to insert an implant with an attached suture through the torn portions of an injured meniscus to hold them together during healing. The implant, which serves as a backstop, is preloaded into the side cutout. The suture is tied to a side of the backstop and passes through a tophat-shaped element that cooperates with the backstop to pull the initially torn-apart portions of the meniscal tissue together when tension is applied to the suture.

In the field of ophthalmic surgery, cryosurgical techniques are known for generating local “freeze burns” to reattach and repair “pin holes” in damaged retinas. These involve the forcible application of intense cold locally from the back of the eye where the repair is to be made. Another known technique for such eye repairs employs fine laser beams directed from the front and through the eyeball to generate local internal burns that heal to reattach torn retinas. The “burn”, no matter how it is generated, by itself causes the burned, i.e., temperature-damaged, tissues initially to stick to each other. The internal pressure of the vitreous material within the affected eyeball thereafter continually acts to hold the “burned” tissues in firm contact with each other while they heal and form a shared “fused scar” to become permanently attached thereby.

No prior art is known that teaches or even suggests either an apparatus or a method to repair a prolapsed vagina, without requiring the making of any vaginal or abdominal incisions, by utilizing deliberately fused scar tissue to permanently attach the vagina in a correct disposition to adjacent support tissue at a plurality of support locations within a patient's pelvis. The present invention, in its various embodiments as disclosed herein, thus provides a simple, minimally invasive, safe, inexpensive solution to this long felt need. These features make the present invention particularly suitable for the elderly, medically fragile, patient who would not be able to safely tolerate currently available techniques.

### SUMMARY OF THE INVENTION

It is a principal object of this invention to provide a simple, minimally invasive and inexpensive apparatus for effecting vaginal prolapse repair without vaginal or abdominal incisions.

Another object is to provide a simple, minimally invasive and inexpensive apparatus by which a surgeon can quickly, relatively painlessly, affordably, safely and permanently repair a prolapsed vagina with any displaced internal organs repositioned in their correct locations within a female patient.

These and other related objects of this invention are realized by providing an apparatus for repairing vaginal prolapse in a female patient, comprising:
an elongate hollow element, having a sharp distal end and a proximate end, of a stiffness and length selected to permit, without incision, insertion of the distal end from within the patient's correctly repositioned vagina through a wall of the vagina and to a selected depth into selected support tissue;
a barbed element having a front end, a rear end and rearwardly-directed barbs, positioned inside the hollow element adjacent the distal end thereof with at least some of the barbs disposed to engage with the selected support tissue immediately upon insertion of the distal end of the hollow element for location of the barbed ele in the support tissue; and
a predetermined length of suture material, connected to the barbed element so as to extend through the vaginal wall following said location of the barbed element in the support tissue.

Another object of this invention is to provide a method by which a surgeon can form a permanent bond between a support tissue and a supported tissue within a patient's body by deliberately forming a fused scar between them at a selected location.

This object is realized by providing a method of forming a permanent local bond between a living support tissue and an adjacent living supported tissue within a patient's body, comprising the step of:
deliberately forming a fused scar between a physically distressed surface portion of the support tissue and a contacting physically distressed surface portion of the supported tissue at a selected location.

A related object of this invention is to provide a method of repairing vaginal prolapse in a patient without the need for forming any vaginal or abdominal incisions.

This object is realized by providing a method comprising the step of:
causing scar fusion between the patient's correctly repositioned vagina and selected support tissue within the patient's pelvis.

These and other related aspects and benefits of the disclosed invention will be better understood from the detailed description provided below with appropriate reference to the appended drawing figures and claims.

### BRIEF DESCRIPTION OF THE DRAWINGS

FIGS. 1A, 1B and 1C respectively show a prior art anchor element with two attached sutures, a stylet, and a delivery needle that are operable in combination, through a vaginal incision, to locate the anchor into the rectus abdominus of a female patient in a procedure for correcting urinary incontinence.

FIGS. 2A and 2B show two other prior art anchor structures.

FIG. 3 is a perspective view of the inside of a female pelvis, from above, to explain the relative locations and interrelationships between a prolapsed vagina and various important structures to some of which the repositioned vagina can be reattached with the teaching of this invention.

FIG. 4 is a perspective view of the inside of a female pelvis, from above, showing how the correctly repositioned vaginal vault may be attached at a plurality of locations to the sacrospinous ligament according to this invention.

FIG. 5 is a sectional close-up of two pairs of anchors, and respective sutures attached thereto, as they would be deployed according to this invention to permanently attach a correctly repositioned vagina to effect prolapse repair.

FIG. 6 is an axial cross-sectional view of the distal end of a preferred embodiment of the apparatus prior to placement of an anchor element in support tissue.

FIG. 7 is an axial cross-sectional view of the distal end of a second preferred embodiment of the apparatus following insertion thereof into either support tissue or supported tissue but before an anchor is finally located in support tissue.

FIG. 8 is a close-up, partially cross-sectional, view to clarify the condition of the supported and support tissues as an anchor element with its attached suture is being located in the support tissue according to this invention.

### DETAILED DESCRIPTION OF THE PREFERRED EMBODIMENTS

Some of the cooperating elements of the apparatus according to the first preferred embodiments of this invention, in certain respects, are structurally similar to those employed in known devices. These aspects are clearly explained below.

The first preferred embodiment of the apparatus, 100, as best seen schematically in FIG. 4, comprises an elongate needle-shaped body 102 formed to have a sharp-edged distal end 104. This distal end 104 is preferably cut at an angle, e.g., about 30-60 degrees relative to the axis of body 102, such that its sharp extreme distal point 106 can easily penetrate selected tissue in a patient's body precisely at a location chosen by the user—presumably a qualified surgeon. Distal end 104 may also be formed to have a short longitudinal slot 108 sized to comfortably accommodate a suture element, and should be provided with smoothly rounded, or at least blunt, edges to avoid unintended severance of the suture.

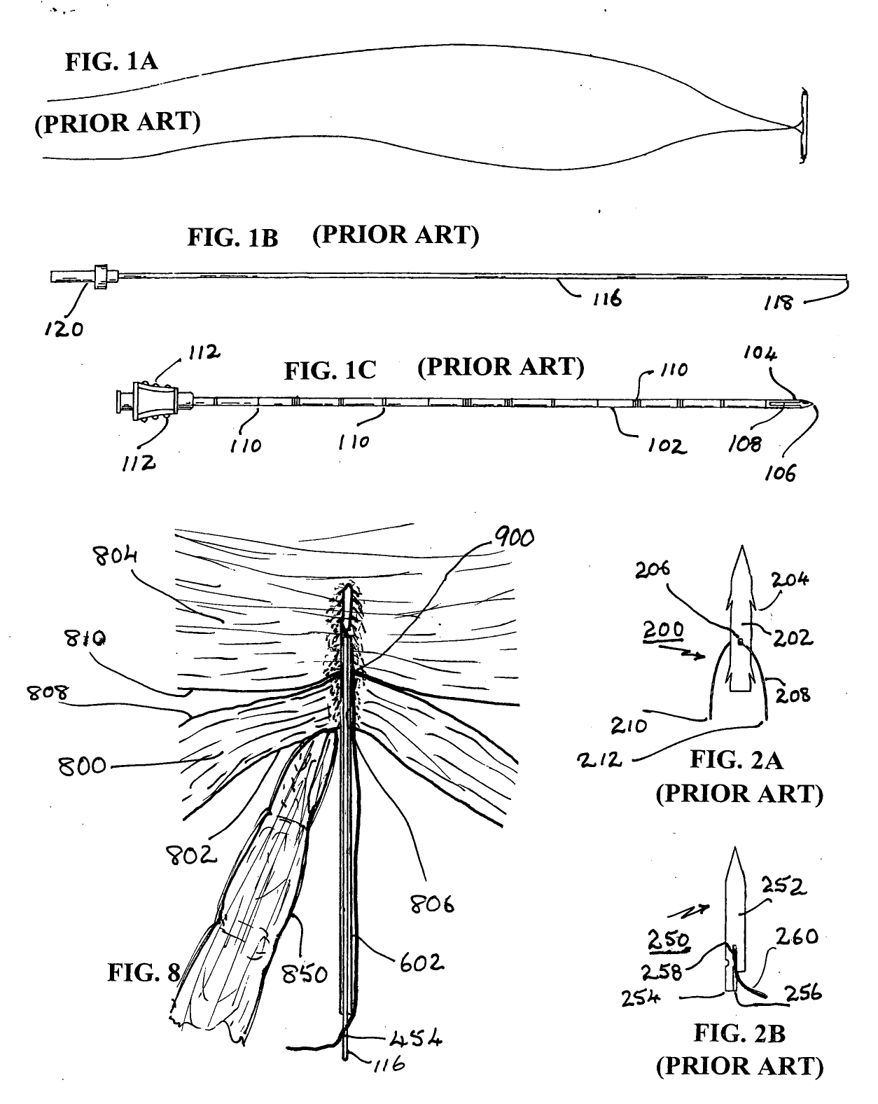

The structure of the present invention as described thus far is comparable in some, but not all, respects to that taught in FIG. 1 of previously described U.S. Pat. No. 6,595,911 to LoVuolo, reproduced as FIG. 1C hereof.

Body 102 preferably comprises a sterilizable and strong but bendable material, e.g., stainless steel. It also may be advantageously provided with a plurality of visually or tactilely perceivable markings, such as 110 and 112 respectively, that will enable the user to readily ascertain its depth relative to the patient's body and its orientation relative to a manually graspable handle such as 114 (best seen in FIG. 4). Note that the same numerals are used to identify counterpart features in both the structure of the present invention and the prior art—solely for the reader's ease of reference. Handle 114 is not limited to the exemplary shape shown in FIG. 4, and may be of any shape and size that best suits a particular user's needs.

An elongate, stiff but flexible stylet element 116, having a forward end 118 and a rear end 120, is sized to be slidably retained and guided longitudinally within the lumen of body 102. Rear end 120 may be made of any suitable shape and size, and may be juxtaposed relative to handle 114 as best suits a user's needs. Such structures, some with safety features to prevent inadvertent relative movements between the body and the stylet, are well known; hence further details are not deemed necessary for persons of ordinary skill in the relevant arts.

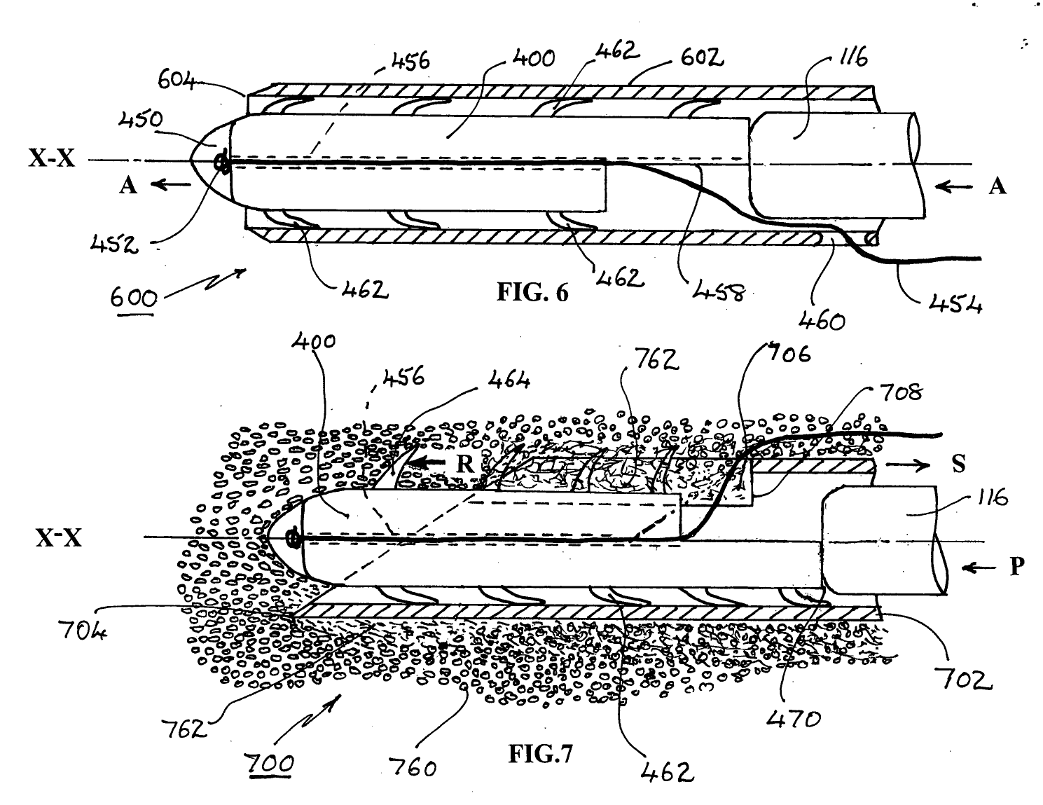

The third and most important structural element of the combination per the first embodiment is an anchor element 400 (best seen in FIGS. 6 and 7) that is locatable by the previously described body 102 and stylet 116 to support a suture 454 that in turn will support the relocated vagina. LoVuolo teaches an anchor element 150 (best seen in FIG. 1) that is partially tubular and has two similar sutures 152, 152 extending approximately centrally therefrom. This anchor is entirely different from the one employed in the present invention.

The teaching of LoVuolo, as it relates to these structures, is hereby incorporated herein by reference.

A detailed description the anchor element 400 per the preferred embodiments is provided below, following a brief discussion of certain features of other known anchors that differ in various key respects.

Two known anchors 200 and 250, as disclosed in FIGS. 17 and 16 of previously described U.S. Pat. No. 6,334,446 to Beyar, are shown respectively in counterpart reproduced FIGS. 2A and 2B hereof. Anchor 200 teaches a sharply pointed body 202 provided with a plurality of rearwardly-oriented barbs 204 and a transverse aperture 206 through which is freely disposed a length of suture 208 that has two free ends 210 and 212. Given that anchor 200 per Beyar is to be forcibly implanted into a patient's bone, a very hard tissue, these barbs 204 clearly cannot be formed to be flexible either during or immediately following implantation. After implanting such an anchor 200, the user is obviously free to move the suture 208 relative to body 202, within aperture 206, as needed. The two ends 210 and 212, equally obviously, are therefore individually usable at the user's discretion. Anchor 250, best seen in FIG. 2B, by contrast has a simple, unbarbed, forwardly-pointed body 252, with a rear end 254 provided with a central slot or through hole 256 inside which is immovably affixed a first end 258 of a suture 260. A portion of the end 254 is cut out so that, when there is forcible contact by a stylet or some other element thereat, there will not be a pinching and perhaps severance of the suture during application of the anchor. The description of these and other types of anchors in Beyar is very sparse, and there is no suggestion that there is any merit in adding barbs to anchor 250, in making the barbs of anchor 200 flexible, or even of making the barbs of an absorbable material.

The teaching of Beyar, to the extent it relates to these structures, is hereby incorporated herein by reference.

It is considered that a basic description of the patient's body structures at issue may be helpful to the reader at this stage in understanding how the invention operates and how it is best used.

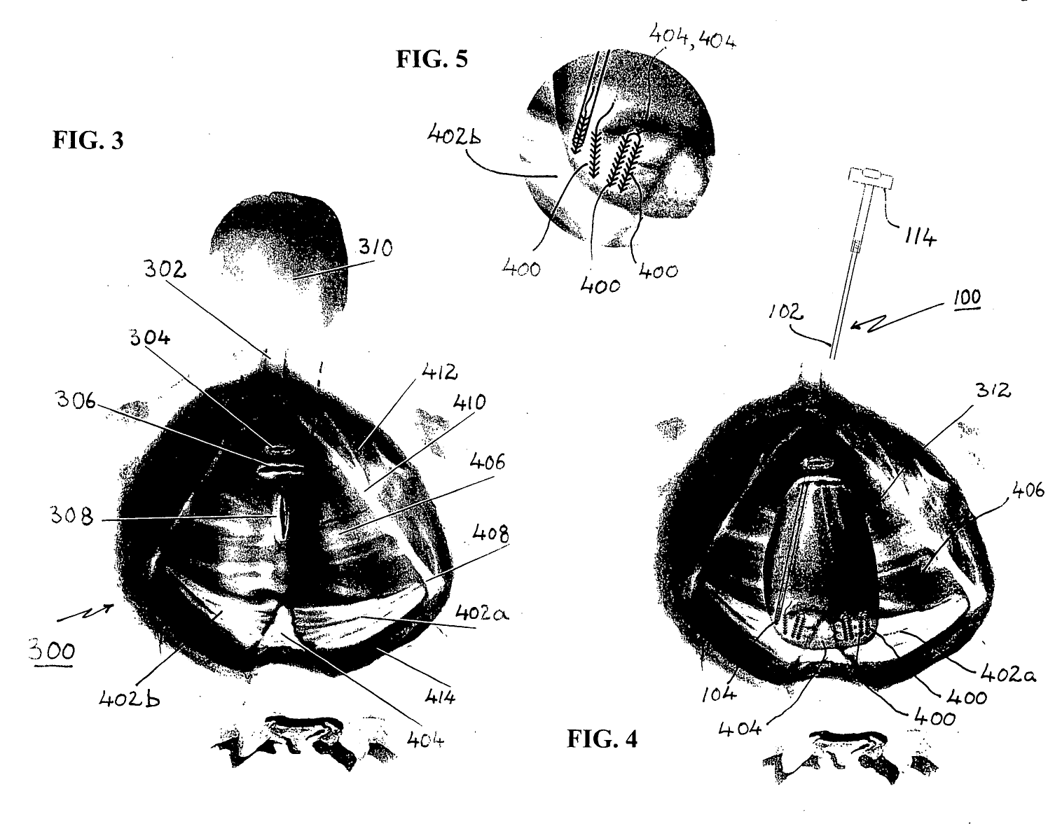

As best understood with reference to FIG. 3, the female pelvis 300 has behind the pubis symphysis 302 a urethral opening 304, a vaginal opening 306 and an anal opening 308. Under normal conditions, urine is voided from the bladder (not visible in this view) via the urethra through urethral opening 304. The uterus (also not visible in this view) normally is situated in the lower pelvis with only the cervix (the lowest part of the uterus) protruding into the upper vagina. However, when she suffers a vaginal prolapse the patient's bladder and/or the uterus may pass out of her pelvic cavity through her vagina and extend well outside the vaginal opening 310, so that they all then depend outside the female's pelvis as schematically indicated in FIG. 3.

The solution to this serious problem initially is to gently but firmly push the displaced organs back into the pelvic cavity through vaginal opening 306 and revert displaced vaginal vault 310 back to its normal well-supported position. After this is accomplished, it is necessary to ensure that the repositioned vaginal vault 312 (best seen in FIG. 4) is secured in its proper place so as to retain the bladder, uterus, rectum or prolapsed vaginal vault in their respective proper places as well.

To do this with the present invention, as best understood with reference to FIGS. 4 and 5, pairs of adjacently located barbed anchor elements 400, 400 are forcibly pushed through the wall of repositioned vaginal vault 312 into selected support tissue such as sacrospinous ligament 402a. Respective sutures 404, 404 that are attached at their distal ends to the rear ends of anchors 400, 400 are left extending through the wall of the vaginal vault 312 into the vagina. They are then tied to each other inside the vaginal vault 312 through the vagina to hold the vaginal wall, i.e., the supported tissue, in firm and intimate surface-to-surface contact with the selected supporting tissue 402a. It may also be appropriate in the surgeon's judgment to make such attachments elsewhere, e.g., with the sacrospinous ligament 402b on the opposite side of the sacrum 404, the levator muscle 406, and/or the ileococcygeus fascia (not visible in the views per FIGS. 4 and 5), the white line 410, or the faschia over the obturator internus muscle 412 that normally provides upward support to the organs of interest. Based on the extent and type of prolapse, anchors may be placed in any of these support structures to facilitate a durable repair. Other structures shown in FIG. 4 are the ischial spine 408 and the piriformis muscle 414. The surgeon will also have to exercise judgment in determining exactly how many pairs of anchors to deploy in each of the selected support tissues. The placement of the anchors 400, 400 is done with the previously described apparatus 100, as detailed below.

As best understood with reference to the partial sectional view of FIG. 6, in a first preferred embodiment 600 of this invention the distal end of elongate needle-shaped body 602 has a sharp bevel-edged distal end 604 with which it can puncture, by movement in the direction of arrow “A”, into and/or through the supporting tissue and the supported tissue. Anchor element 400 has an elongate cylindrical body with a diameter smaller than the internal bore diameter of body 602 within which it is initially held just inboard of sharp-edged end 604. It may optionally, but not necessarily, have a rounded end, preferably with a distal notch or recess 450 sized to receive and hold distal end 452 of a suitable length of suture 454. A knot may be formed at end 452 or some other mechanism employed to retain end 454 in anchor element 400 such that the suture itself extends centrally, e.g., via an axial bore 456, toward the rear of anchor 400.

Anchor element 400, like anchor element 250 per Beyar (see FIG. 2A), preferably has a notched rear end portion 458 that serves to avoid pressure on suture 454 from the front end of stylet 116 which, for the same reason, may be provided a rounded end edge. Such a pressure could arise if the body 602 is held stationary and stylet 116 is pushed forward relative to it in order to expel anchor element 400 in the direction of arrow “A”. In this first preferred embodiment, a small hole 460 is provided in needle-shaped body 602, inboard of its distal end 604, to permit safe extension of 454 outside and along body 602 as shown in Fig.6. Hole 460 should not have sharp edges.

Anchor element 400 is preferably made of a known firm but flexible and biocompatible material and is formed to have a plurality of rearwardly-oriented external barbs that are sized so that they must flex radially inwards when contained within the internal bore of body 602. The material of anchor element 400 may be chosen to be absorbable or nonabsorbable, for reasons discussed later.

With the structure described in the immediately preceding paragraphs, with body 602 and the stylet 116 held immovable relative to each other, with anchor element 44 held in front of the stylet, the surgeon can apply the distal end of the combination to puncture through the vaginal wall from inside the repositioned vagina. Doing so will cause deliberately inflicted physical, i.e., mechanically induced, distress to the vaginal wall.

As best understood with reference to Fig.8, the surgeon first must palpate with a finger 850 applied to the inside surface 802 of vaginal wall 800 to locate a suitable support tissue 804, select a specific point of entry 806 for the puncture, and then carefully guide the sharp-edged distal end 604 of needle-shaped body 602 there for the puncturing motion. Further pressure will force the sharp-edged end 604, and thus the anchor element 400 initially just inboard of it, through the inside surface 808 of the vaginal vault and then through surface 810 of support tissue 804 to a selected depth therein. This will be facilitated by reference to the previously described markings outside and along the length of needle body 602.

Rearward movement of needle body 602, i.e., opposite to the direction of arrow “A”, and relative to stationarily held anchor element 400 and stylet 116, now will cause anchor element 400 to stay in place, at the selected depth inside support tissue 804, by engagement of the released elastic barbs 462 therewith. This is preferable to pushing the stylet forward relative to body 602 because doing so may cause the anchor element to be driven to a less definite depth into the supporting tissue. This may in practice amount to the exercise of surgical technique and finesse. Once barbs 462 engage with the supporting tissue, body 602 and stylet 116 are withdrawn together, first out of the supporting tissue 804, then through the supported tissue of vaginal wall 800, and finally entirely out of the vagina. Suture 454 meanwhile will have continued to extend out of hole 460 along and outside body 602 during the puncturing insertion, and will now be held at its distal end by anchor element 400 to remain extended through the punctures in both tissues and into the vaginal vault. This process is repeated at a second insertion point close to the previous one. The two adjacent sutures are then tied to each other after they are drawn tightly enough to bring the supported and supporting tissues in firm and intimate contact at the respective punctures.

The second preferred embodiment 700 differs from the first one in two significant particulars. First, best understood with reference to FIG. 7, the needle-like body 702 differs from body 602 in that it has an angled sharp-edged distal end ending in a distal point 704. It is provided with a longitudinal slot that is sized so that when anchor element 400 is initially disposed within the slotted region the proximate end 470 of anchor element extends past the proximate end 708 of slot 706. The width of slot 706 must be greater than the base width of the barbs 462, 464 but smaller than the diameter of anchor element 400. This will ensure that at least some of the barbs, 464, extend out of slot 706 beyond the outer surface of body 702 even before any tissue is penetrated; and it will also ensure that anchor element 400 will not be forced sideways out of body 702 by the penetrated tissues as the puncturing process proceeds.

Most of the barbs 462, if made of preferred stiff but flexible material, will remain inwardly bent to fit within the inner lumen of body 702 until anchor element 400 is released therefrom as the latter is withdrawn together with stylet 116. Such materials are known and used in a variety of implant structures—both absorbable and nonabsorbable. Even if the material is inflexible, the compliance of the body tissues contacting the exposed barbs via the slot will cause the barbs to become engaged with any tissue penetrated by the distal end of body 702. If the anchor element is made of a stiff material, such that the barbs cannot flex, the diamertically outermost dimension of the anchor element must be selected to permit slidable but close containment thereof inside the body 702.

What is particularly noteworthy about this second preferred embodiment 700 is that no relative motion between body 702 and stylet 116 is required because as soon as the barbs 464 are pushed into either the supported tissue or the supporting tissue they will engage therewith and cannot easily be drawn back rearwardly. If it is deemed necessary, the stylet can be pushed forward in the direction of arrow “P” and/or the body 702 may be slid relatively rearward in the direction of arrow “S” to facilitate movement of anchor element 400 outward. Forward motion of the combined elements of the apparatus, to obtain the desired puncturing of both tissues, is of course always possible because the barbs are both flexible and rearwardly-oriented.

As best understood with careful reference to FIG. 7, as the combination of body 702, anchor element 400 and stylet 116 moves first into the supported tissue (and then through the supporting tissue), there will be physical breakage and tearing of initially intact tissue cells 760 (shown as generally round in cross-section). The torn cells will release their liquid contents and there will also be local bleeding (shown by short wavy lines). As will be appreciated, once the body 702 (or 602 if the first embodiment is considered) is withdrawn, the torn cells of supporting tissue 804 and supported tissue 800 will be in intimate contact—especially at the respective punctures left in each, i.e., at 900 (best seen in Fig.8). The suture extending through and keeping the punctures aligned will ensure that as healing proceeds the replacement cells of the two tissues will fuse together as they form a fused scar at and immediately surrounding 900.

With the contemplated technique, the surgeon will generate numerous pairs of such adjacent fused scars (rather like plural pairs of spot welds between two contacting metal surfaces in engineering applications) that will hold the supported tissue of the vaginal vault very firmly to the supporting tissue. The surgeon must exercise judgment, based on the patient's condition and needs, on the number of such fused scar pairs, the choice of supporting tissues, and the specific locations for the fused scars.

The healing process resulting in long lasting durable scarification will take approximately four weeks, and with the use of appropriate antibiotics and care there should be little risk of infection. The anchor elements may conveniently be a fraction of an inch in length, and may be left in place permanently since they are buried in the anchoring support tissue. They therefore do not have to be absorbable. The sutures, on the other hand, probably are best made of absorbable material that will disappear in about six to eight weeks since the knot tying each pair of sutures is left in the vaginal lumen, i.e., after the fused scars have become well established and are capable of holding the desired bonds between the supported and supporting tissues. It will, of course, be appreciated that if the anchor elements are made of absorbable material they will eventually disappear together with any absorbable sutures. There are thus a variety of choices available in selecting from among known materials for these elements.

It is intended that the present invention comprehend all obvious variations and modifications of the disclosed structures and methods and that it be limited solely by the claims presented herein.

1. Apparatus for repairing vaginal prolapse in a female patient, comprising:
an elongate hollow element having a sharp distal end and a proximate end, of a stiffness and length both selected to permit, without incision, insertion of the distal end from within the patient's correctly repositioned vagina through a wall of the vagina and to a selected depth into selected support tissue;
a barbed element having a front end, a rear end and rearwardly-directed barbs, positioned inside the hollow element adjacent the distal end thereof with at least some of the barbs disposed to engage with the support tissue immediately upon insertion of the distal end of the hollow element for location of the barbed element in the support tissue; and
a predetermined length of suture material, connected to the barbed element so as to extend through the vaginal wall following said location of the barbed element in the support tissue.

2. The apparatus according to claim 1, further comprising;
a manually graspable handle attached to the elongate hollow element adjacent the proximate end thereof.

3. The apparatus according to claim 1, wherein:
the elongate hollow element is formed to have at least one of a longitudinal slot and a smooth-edged side aperture adjacent the distal end.

4. The apparatus according to claim 1, wherein:
at least one of the barbed element and the suture element comprises an absorbable material.

5. The apparatus according to claim 1, further comprising:
a push element slidable within the first element to push the elongate hollow element rearwardly away from the barbed element following location of the barbed element in the support tissue.

6. Apparatus for generating a permanent local bond between a living support tissue and an adjacent living supported tissue, comprising:
a tubular element of a predetermined first length, having a distal end and a proximate end, the distal end being formed to be forcibly insertable through the supported tissue and into the support tissue;
an anchor element having a front end and a rear end, sized to be slidably retained at least partially in the tubular element adjacent the distal end thereof, provided with a plurality of rearward oriented barbs shaped and disposed to resist rearward movement out of the support tissue after placement of the anchor element therein;
an elongate element, having a forward end and a back end, sized to be slidably guided within the tubular element, with the forward end disposed behind the rear end of the anchor element so as to facilitate movement of the tubular element rearwardly relative to the anchor element for location of the anchor element in the support tissue; and
a suture element of a predetermined second length extending between first and second ends, attached at the first end to the anchor element so as to extend from said rear end thereof upon said placement of the anchor element in the support tissue.

7. The apparatus according to claim 6, further comprising:
a manually graspable handle attached to the tubular element adjacent the proximate end thereof.

8. The apparatus according to claim 6, wherein.
the tubular element is formed to have at least one of a longitudinal slot at the distal end and a smooth-edged side aperture adjacent the distal end.

9. The apparatus according to claim 6, wherein:
at least one of the anchor element and the suture element comprises an absorbable material.

10. A method of forming a permanent local bond between a living support tissue and an adjacent living supported tissue, comprising the step of:
deliberately forming a fused scar between a physically distressed surface portion of the support tissue and a contacting physically distressed surface portion of the supported tissue at a selected location within a patient.

11. The method according to claim 10, wherein:
the physically distressed portions of the supported tissue and the support tissue comprise aligned local punctures.

12. A method of repairing vaginal prolapse in a female patient, comprising the step of:
causing scar fusion between the patient's correctly repositioned vagina and selected support tissue within the patient's pelvis.

13. A method of repairing vaginal prolapse in a patient, comprising the steps of:
restoring any of the patient's organs that have become displaced back to their respective proper positions;
repositioning the vagina to its proper position within the patient;
palpating from within the repositioned vagina to locate a suitable support tissue location;
inserting a barbed first anchor element having a rearwardly extending first suture element, from the inside of the repositioned vagina and through the wall of the vagina at a first insertion point thereat and into a first anchor position within the selected support tissue location, so that a proximate end portion of the first suture element extends into the vagina at the first insertion point;
inserting a second barbed anchor element having a rearwardly extending second suture element, from the inside of the repositioned vagina and through the wall of the vagina at a second insertion point adjacent to the first insertion point and into a second anchor position adjacent to the first anchor position also within the selected support tissue location, so that a proximate end portion of the second suture element extends into the vagina at the second insertion point; and
securing the proximate end portions of the first and second suture elements to each other so that the first and second insertion points and the corresponding first and second anchor positions are respectively aligned in firm physical contact and so retained during healing until corresponding first and second fusion scars are formed corresponding to the first and second anchor positions to thereby permanently bond the vaginal wall to the support tissue.

14. The method according to claim 13, comprising the further steps of:
forming additional similar pairs of fusion scars to thereby form corresponding additional permanent bonds between the vaginal wall and selected support tissue.

15. The method according to claim 13, wherein:
the suture elements comprise an absorbable material.

16. The method according to claim 13, wherein:
the barbed anchor elements comprise an absorbable material.

17. The method according to claim 13, wherein:
the barbed anchor elements comprise a nonabsorbable material.

18. The method according to claim 13, wherein:
all of the barbed anchor elements and all the suture elements comprise an absorbable material.

19. The method according to claim 13, wherein:
the support tissue location is selected from a group of tissue locations consisting of the patient's sacrospinous ligament complex or the ileococcygeus muscle adjacent the top of the vaginal vault, the arcus tendinous fascia pelvis on either side of the anterior vaginal wall, and the levator muscle adjacent the posterior vaginal wall.

20. The method according to claim 19, comprising the further steps of:
forming additional fusion scars to thereby form corresponding additional permanent bonds between the vaginal wall and other selected support tissue locations.

21. The method according to claim 19, wherein:
the suture elements comprise an absorbable material.

22. The method according to claim 19, wherein:
the barbed anchor elements comprise an absorbable material.

23. The method according to claim 19, wherein:
the barbed anchor elements comprise a nonabsorbable material.
